(19) United States
(12) Patent Application Publication
(10) Pub. No.: US 2003/0225163 A1
(43) Pub. Date: Dec. 4, 2003

(54) Sulfonamide derivatives

(76) Inventors: Macklin Brian Arnold, Morgantown, IN (US); Thomas John Bleisch, Noblesville, IN (US); George William Cuff, Indianapolis, IN (US); Paul Leslie Ornstein, Carmel, IN (US); Dennis Michael Zimmerman, Zionsville, IN (US)

Correspondence Address: ELI LILLY AND COMPANY, PATENT DIVISION, P.O. BOX 6288, INDIANAPOLIS, IN, 46206-6288, US

(21) Appl. No.: 10/258,159

(22) PCT Filed: May 4, 2001
(86) PCT No.: PCT/US01/11747

(51) Int. Cl. A61K 31/18; C07C 311/01

(52) U.S. Cl. 514/601; 564/97

(57) ABSTRACT

The present invention relates to a compound of formula (I) or a pharmaceutically acceptable salt thereof which is useful for the treatment of conditions associated with glutamate hypofunction, such as psychiatric and neurological disorders.

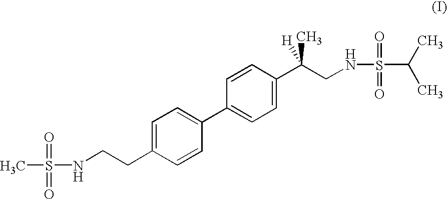

In the mammalian central nervous system (CNS), the transmission of nerve impulses is controlled by the interaction between a neurotransmitter, that is released by a sending neuron, and a surface receptor on a receiving neuron, which causes excitation of this receiving neuron. L-Glutamate, which is the most abundant neurotransmitter in the CNS, mediates the major excitatory pathway in mammals, and is referred to as an excitatory amino acid (EAA). The receptors that respond to glutamate are called excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general types. Receptors that are directly coupled to the opening of cation channels in the cell membrane of the neurons are termed “ionotropic”. This type of receptor has been subdivided into at least three subtypes, which are defined by the depolarizing actions of the selective agonists N-methyl-D-aspartate (NMDA), alpha-amino-3-hydroxy-5-methylisoxazole-4-propionic acid (AMPA), and kainic acid (KA). The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in c-AMP formation, and changes in ion channel function. Schoepp and Conn, Trends in Pharmacol. Sci., 14, 13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15,41 (1990).

AMPA receptors are assembled from four protein sub-units known as GluR1 to GluR4, while kainic acid receptors are assembled from the sub-units GluR5 to GluR7, and KA-1 and KA-2. Wong and Mayer, Molecular Pharmacology 44: 505-510, 1993. It is not yet known how these sub-units are combined in the natural state. However, the structures of certain human variants of each sub-unit have been elucidated, and cell lines expressing individual sub-unit variants have been cloned and incorporated into test systems designed to identify compounds which bind to or interact with them, and hence which may modulate their function. Thus, European patent application, publication number EP-A2-0574257 discloses the human sub-unit variants GluR1B, GluR2B, GluR3A and GluR3B. European patent application, publication number EP-A1-0583917 discloses the human sub-unit variant GluR4B.

One distinctive property of AMPA and kainic acid receptors is their rapid deactivation and desensitization to glutamate. Yamada and Tang, The Journal of Neuroscience, September 1993, 13(9): 3904-3915 and Kathryn M. Partin, J. Neuroscience, Nov. 1, 1996, 16(21): 6634-6647.

It is known that the rapid desensitization and deactivation of AMPA and/or kainic acid receptors to glutamate may be inhibited using certain compounds. This action of these compounds is often referred to in the alternative as “potentiation” of the receptors. One such compound, which selectively potentiates AMPA receptor function, is cyclothiazide. Partin et al., Neuron. Vol. 11, 1069-1082, 1993.

International Patent Application Publication WO 98/33496 published Aug. 6, 1998 discloses certain sulfonamide derivatives which are useful, for example, for treating psychiatric and neurological disorders, for example cognitive disorders; neuro-degenerative disorders such as Alzheimer's disease; age-related dementias; age-induced memory impairment; movement disorders such as tardive dyskinesia, Hungtington's chorea, myoclonus, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, and drug-induced psychosis.

The present invention provides a compound of formula I:

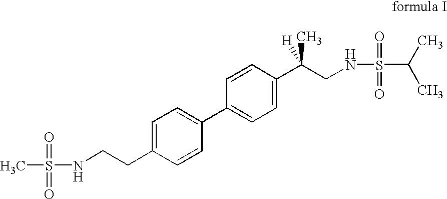

or a pharmaceutically acceptable salt thereof.

The present invention further provides a method of potentiating glutamate receptor function in a patient which comprises administering to said patient an effective amount of a compound of formula I.

In addition, the present invention provides a method of treating depression in a patient comprising administering to said patient an effective amount of a compound of formula I.

The present invention further provides a method of treating schizophrenia in a patient comprising administering to said patient an effective amount of a compound of formula I.

Furthermore, the present invention provides a method of treating cognitive is disorders in a patient comprising administering to said patient an effective amount of a compound of formula I.

The invention further provides pharmaceutical compositions of compounds of formula I, including the hydrates thereof, comprising, as an active ingredient, a compound of formula I in combination with a pharmaceutically acceptable carrier, diluent or excipient.

This invention also encompasses novel intermediates, and processes for the synthesis of the compounds of formula I.

In addition, the present invention provides the use of a compound of formula I or a pharmaceutically acceptable salt thereof for potentiating glutamate receptor function.

According to another aspect, the present invention provides the use of a compound of formula I for the manufacture of a medicament for potentiating glutamate receptor function.

The present invention further provides an article of manufacture comprising packaging material and a compound of formula I or a pharmaceutically acceptable salt thereof contained within said packaging material, wherein said packaging material comprises a label which indicates that said compound of formula I can be used for treating at least one of the following; Alzheimer's disease, schizophrenia, cognitive deficits associated with schizophrenia, depression, and cognitive disorders.

The present invention further provides a pharmaceutical composition prepared by a process comprising dissolving {(2R)-2-[4-(4-{2-[(methylsulfonyl)amino]ethyl}phenyl)phenyl]propyl}[(methylethyl)sulfonyl]amine in a suitable polyethylene glycol in liquid form, and then cooling the solution to room temperature.

### DETAILED DESCRIPTION OF THE INVENTION

In this specification, the term “potentiating glutamate receptor function” refers to any increased responsiveness of glutamate receptors, for example AMPA receptors, to glutamate or an agonist, and includes but is not limited to inhibition of rapid desensitization or deactivation of AMPA receptors to glutamate.

A wide variety of conditions may be treated or prevented by the compounds of formula I and their pharmaceutically acceptable salts through their action as potentiators of glutamate receptor function. Such conditions include those associated with glutamate hypofunction, such as psychiatric and neurological disorders, for example cognitive disorders; neuro-degenerative disorders such as Alzheimer's disease; age-related dementias; age-induced memory impairment; movement disorders such as tardive dyskinesia, Hungtington's chorea, myoclonus, dystonia, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, and drug-induced psychosis. In addition, the compounds of formula I are useful for treating sexual dysfunction. The compounds of formula I may also be useful for improving memory (both short term and long term) and learning ability. The present invention provides the use of compounds of formula I for the treatment of each of these conditions.

As used herein the name “{(2R)-2-[4-(4-{2-[(methylsulfonyl)aminoethyl}phenyl)phenyl]propyl}[(methylethyl)sulfonyl]amine” refers to the compound of formula I:

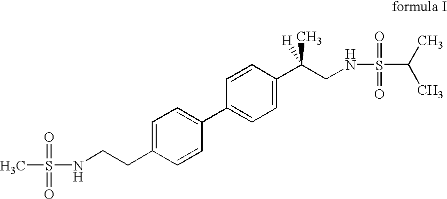

As used herein the name “(methylsulfonyl){2-[4-(4-{2-[(methylsulfonyl)amino]ethyl}phenyl)phenyl]ethyl}amine” refers to achiral dimer of the following structure:

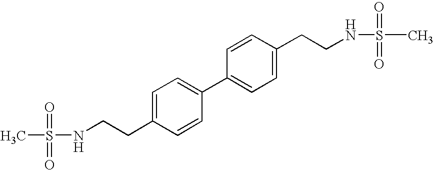

A used herein the name “((2R)-2-{4-[4-((1R)-1-methyl-2-{[(methylethyl)sulfonyl]amino}ethyl)phenyl]phenyl}propyl)[(methylethyl)sulfonyl]amine” refers to chiral dimer of the following structure:

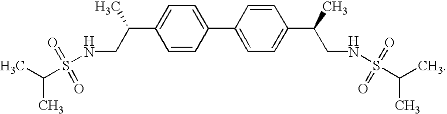

The present invention includes the pharmaceutically acceptable salts of the compounds defined by formula I. The term “pharmaceutically acceptable salt” as used herein, refers to salts of the compounds of the above formula which are substantially non-toxic to living organisms. Typical pharmaceutically acceptable salts include those salts prepared by reaction of the compounds of the present invention with a pharmaceutically acceptable organic or inorganic base. Such salts are known as base addition salts. Such salts include the pharmaceutically acceptable salts listed in Journal of Pharmaceutical Science, 66, 2-19 (1977) which are known to the skilled artisan.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ⅇⅇ = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 70:30 is achieved, the ee with respect to the first enantiomer is 40%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

As used herein the term “Lg” refers to a suitable leaving group. Examples of suitable leaving groups are Cl, Br, and the like.

The compounds of formula I can be prepared, for example, following analogous procedures set forth in International Patent Application Publication WO 98133496 published Aug. 6, 1998 (See Example 51 therein) to prepare the racemate of formula I followed by resolution to provide the desired (R) enantiomer (formula I) or the (S) enantiomer. More specifically, the compounds of formula I can be prepared, for example, following the procedures set forth in Schemes I, II, III, and IIIA. The reagents and starting materials are readily available to one of ordinary skill in the art. All substituents, unless otherwise specified are as previously defined.

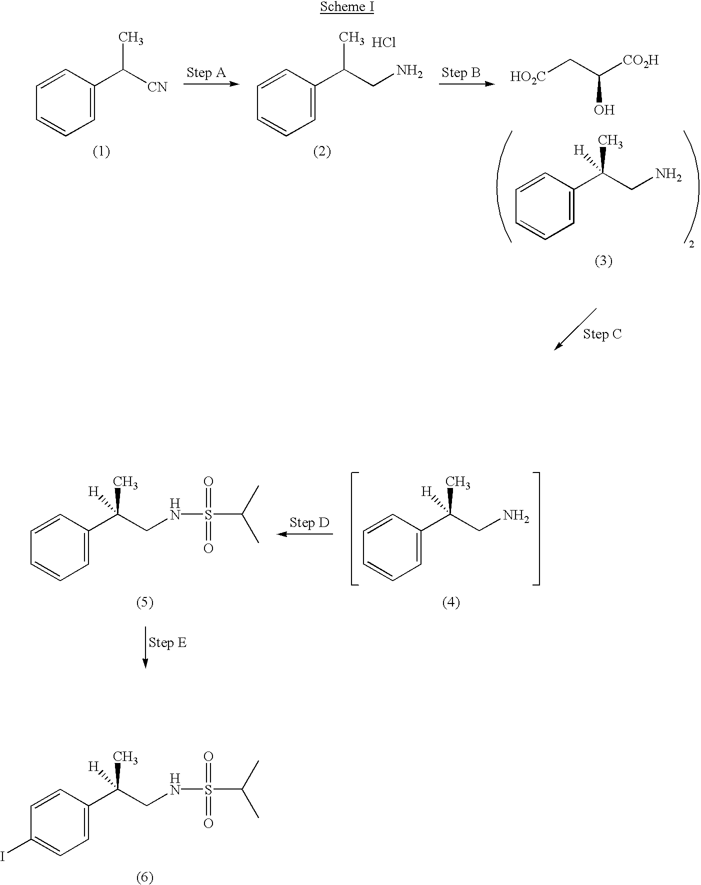

In Scheme I, step A, the nitrile (1) is hydrogenated to provide the primary amine (2) as the HCl salt. For example, nitrile (1) is dissolved in a suitable organic solvent, such as ethanol, treated with a suitable hydrogenation catalyst, such as palladium on carbon, treated with concentrated HCl and placed under hydrogen at a pressure and temperature sufficient to effect reduction of the nitrile (1) to the primary amine (2). The reaction is then filtered and the filtrate concentrated to provide crude primary amine (2) as the HCl salt. This crude material is then purified by techniques well known in the art, such as recrystallization from a suitable solvent.

In Scheme I, step B, the primary amine (2) HCl salt can be treated with a suitable resolving agent to provide the salt (3). For example, the primary amine (2) HCl salt is dissolved in a suitable organic solvent, such as ethanol and treated with about an equivalent of a suitable base, such as sodium hydroxide. The reaction is filtered and the filtrate is treated with a suitable resolving agent, such as L-malic acid. For example, about 0.25 equivalents of L-malic acid in a suitable organic solvent, such as ethanol is added to the filtrate. The solution is then heated to about 75° C. and stirred for about 30 minutes. The solution is then allowed to cool slowly with stirring. The precipitate is then collected by filtration, rinsed with ethanol and dried under vacuum to provide the salt (3). The salt (3) is then suspended in a suitable organic solvent, such as ethanol and water is added. The slurry is heated at reflux until the solids go into solution. The solution is then allowed to cool slowly with stirring for about 8 to 16 hours. The suspension is further cooled to about 0 to 5° C. and the salt (3) is collected by filtration. The salt (3) is then rinsed with ethanol and dried at about 35° C.

In Scheme I, step C, salt (3) is converted to the free base (4) and in Step is D, free base (4) is sulfonylated to provide sulfonamide (5). For example, salt (3) is slurried in a suitable organic solvent, such as methylene chloride and treated with about 2 equivalents of a suitable base, such as aqueous sodium hydroxide. The mixture is stirred for about one hour and the organic phase is separated. The organic phase is then dried, for example by azeotropic distillation with heptane to provide the free base (4). The dried free base (4) in heptane is then treated, for example, with a catalytic amount of 4-dimethylaminopyridine, an excess of triethylamine and methylene chloride is added to provide total dissolution. The solution is cooled to about 5° C. and treated with about one equivalent of a compound of formula Lg-SO₂CH(CH₃)₂, such as isopropylsulfonyl chloride. The reaction is then allowed to warm to room temperature over about 16 hours. The reaction is then cooled to about 8° C. and treated with 2N aqueous HCl. The organic phase is then separated and washed with water, sodium bicarbonate, dried over anhydrous sodium sulfate, filtered, and concentrated under vacuum to provide sulfonamide (5).

In Scheme I, step E, the sulfonamide (5) is iodinated to provide the compound (6). For example, sulfonamide (5) is dissolved in glacial acetic acid and treated with approximately 1.1 equivalents concentrated sulfuric acid. To this solution is added about 0.2 equivalents H₅IO₆ followed by addition of about 0.5 equivalents of iodine. The reaction is then heated to about 60° C. and allowed to stir for about 3 hours. The reaction is then cooled and treated with 10% aqueous NaHSO₃. The mixture is then cooled to about 0° C. to about 5° C. and the resulting solids are collected by filtration and rinsed with water. The solids are then dissolved in a suitable organic solvent, such as MTBE and the solution is rinsed with water, saturated sodium bicarbonate, dried over anhydrous magnesium sulfate, filtered, and partially concentrated under vacuum. A suitable organic solvent, such as heptane is then added with slow stirring until crystallization commences. An additional amount of heptane is added and the suspension is allowed to stir for about 8 hours to about 16 hours. The mixture is then cooled to about 0° C. and the solids are collected by filtration and rinsed with heptane to provide the compound (6).

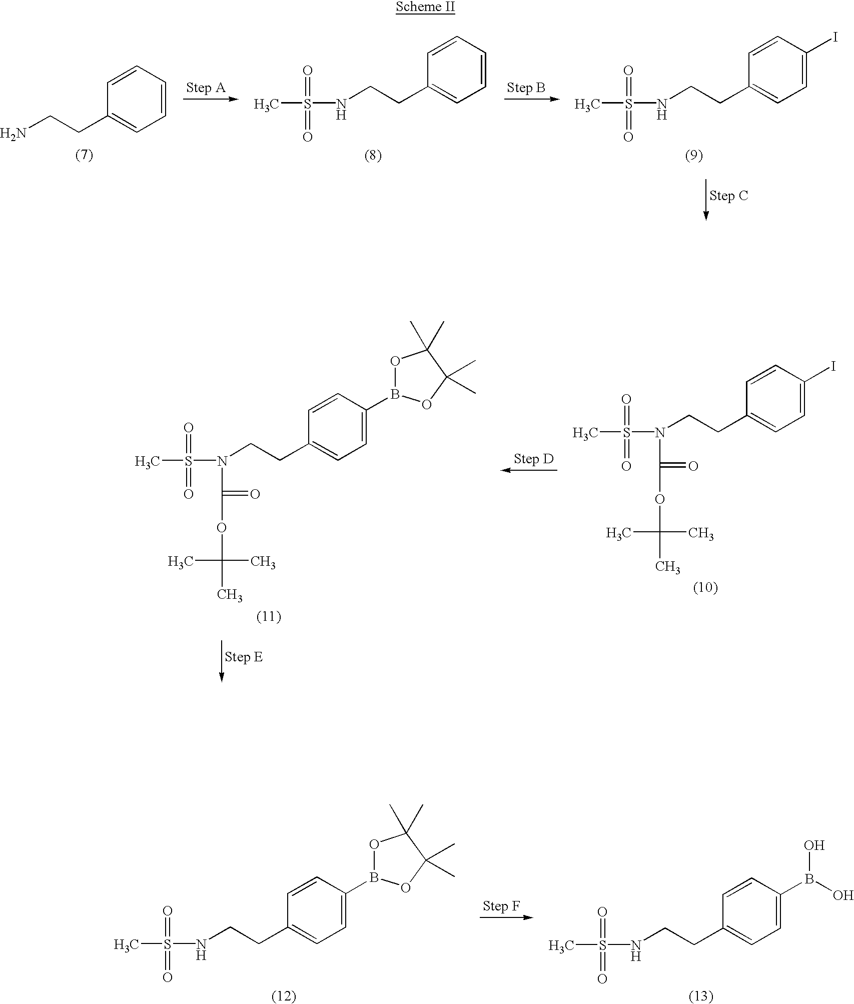

In Scheme II, step A, the primary amine (7) sulfonylated to provide the sulfonamide (8). For example, primary amine (7) is dissolved in a suitable organic solvent, such as methylene chloride and treated with about 1.1 equivalents of triethylamine. The solution is cooled to about 100° C. and treated with about 1.1 equivalents of methanesulfonyl chloride. The solution is then stirred at room temperature for about 1 to 2 hours, washed with 1 N HCl and then concentrated under vacuum to provide sulfonamide (8).

In Scheme II, step B, the sulfonamide (8) is iodinated to provide compound (9). For example, sulfonamide (8) is combined with acetic acid, 95% sulfuric acid and water and then treated with about 0.5 equivalents iodine and about 0.2 equivalents periodic acid. The reaction mixture is heated to about 70° C. to about 75° C. for about 3 hours. The reaction mixture is then allowed to stir at room temperature for about 8 hours to about 16 hours. Then about 2 equivalents of base are added such as sodium hydroxide followed by addition of enough saturated sodium sulfite to decolorize the mixture, resulting in a white suspension. The suspension is cooled to about 15° C. and the solids collected by filtration. The solids are then dissolved in a suitable organic solvent, such as methylene chloride, rinsed with water, and the organic phase concentrated under vacuum to provide the compound (9).

In Scheme II, step C, compound (9) is converted to Boc sulfonamide (10). For example, compound (9) is dissolved in a suitable organic solvent, such as methylene chloride and treated with a catalytic amount of 4-dimethylaminopyridine and about 1.2 equivalents of di-tert-butyl dicarbonate.

The reaction mixture is then allowed to stir at room temperature for about 8 hours to about 16 hours. The reaction is then rinsed with water and the organic phase is partially concentrated under vacuum. A suitable organic solvent is added, such as hexanes and this solution is again rinsed with water. The organic phase is then concentrated under vacuum and hexanes are added producing a precipitate. The solids are collected by filtration and dried under vacuum to provide Boc sulfonamide (10).

In Scheme II, step D, the Boc sulfonamide (10) is subjected to boronation conditions to provide compound (11). For example, the Boc sulfonamide (10) is dissolved in a suitable organic solvent, such as acetonitrile, and treated with excess triethylamine, a catalytic amount of 1,1′-bis(diphenylphosphino) ferrocenedichloropalladium (II)—CH₂Cl₂ complex (2.9 g, 0.0035 mol) and about 1.3 equivalents of pinacolborane. The reaction mixture is allowed to stir at about 70° C. to about 74° C. for about 8 hours. The reaction is then cooled to room temperature and concentrated to a fluid oil. This oil is partitioned between a suitable organic solvent, such as MTBE and water. The organic phase is separated, washed with water and concentrated under vacuum. The residue is partially dissolved in a suitable organic solvent, such as heptane. The heptane solution is filtered through Celite® 521 and the filtrated is concentrated under vacuum to provide an oil. The residue is dissolved in a solvent mixture of acetone and heptane and filtered through Celite® 521. The filtrates are concentrated under vacuum to provide compound (11).

In Scheme II, step E, compound (11) is deprotected to provide the compound (12). For example, compound (11) is dissolved in a suitable organic solvent, such as methylene chloride and treated with excess trifluoroacetic acid. The reaction mixture is cooled to about 5° C. and neutralized with aqueous base, such as aqueous sodium hydroxide to provide a pH of the aqueous phase of about 10.5. The phases are separated and the aqueous phase is extracted with a suitable organic solvent, such as methylene chloride. The organic phase and organic extracts are combined, washed with brine, water, diluted with heptane and concentrated under vacuum to provide a suspension. The solids are collected by filtration, rinsed with pentane, and dried under vacuum to provide compound (12).

In Scheme II, step F, compound (12) is subjected to boron pinacolate cleavage to provide compound (13). For example, compound (12) is combined with 1 N ammonium acetate and excess sodium periodate in a suitable organic solvent, such as acetone. The mixture is stirred for about 8 hours to about 16 hours, and then filtered. The solids are rinsed with acetone. The filtrates are combined and concentrated under vacuum to provide a suspension which is collected by filtration. The collected solid is then suspended in water and treated with aqueous sodium hydroxide to provide a pH of about 12.5. The suspension is then filtered and the filtrate treated with decolorizing carbon. The mixture is then filtered and the filtrate is diluted with sulfuric acid until the pH reaches about 5.0. The resulting precipitate is collected by filtration and dried under vacuum to provide compound (13).

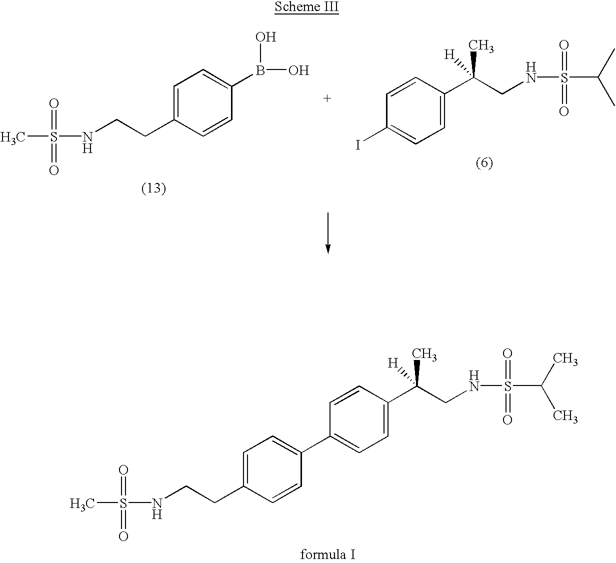

In Scheme III the compound (13) is coupled to compound (6) to provide the compound of formula I. For example, an aqueous solution of potassium formate is prepared by combining water, potassium hydroxide and one equivalent of 98% formic acid. To this solution is then added about 0.2 equivalents potassium carbonate, about 1.8 equivalents of compound (13), and about 2.0 equivalents of compound (6) in a suitable organic solvent, such as n-propanol. It is understood that the above components, including the suitable organic solvent, can be added in any order to the aqueous potassium formate solution. To this mixture, which has been deoxygenated and place under nitrogen, is added a catalytic amount of palladium black, and again the mixture is deoxygenated and placed under nitrogen. The mixture is then heated at about 88° C. for about 8 hours to about 16 hours. The reaction mixture is then cooled and diluted with a suitable organic solvent, such as ethyl acetate. It is then filtered through Celite®, the filtrate is concentrated under vacuum, and the residue partitioned between ethyl acetate and water. The organic phase is separated, concentrated under vacuum, and the residue recrystallized from a suitable solvent mixture, such as acetone/water to provide the compound of formula I.

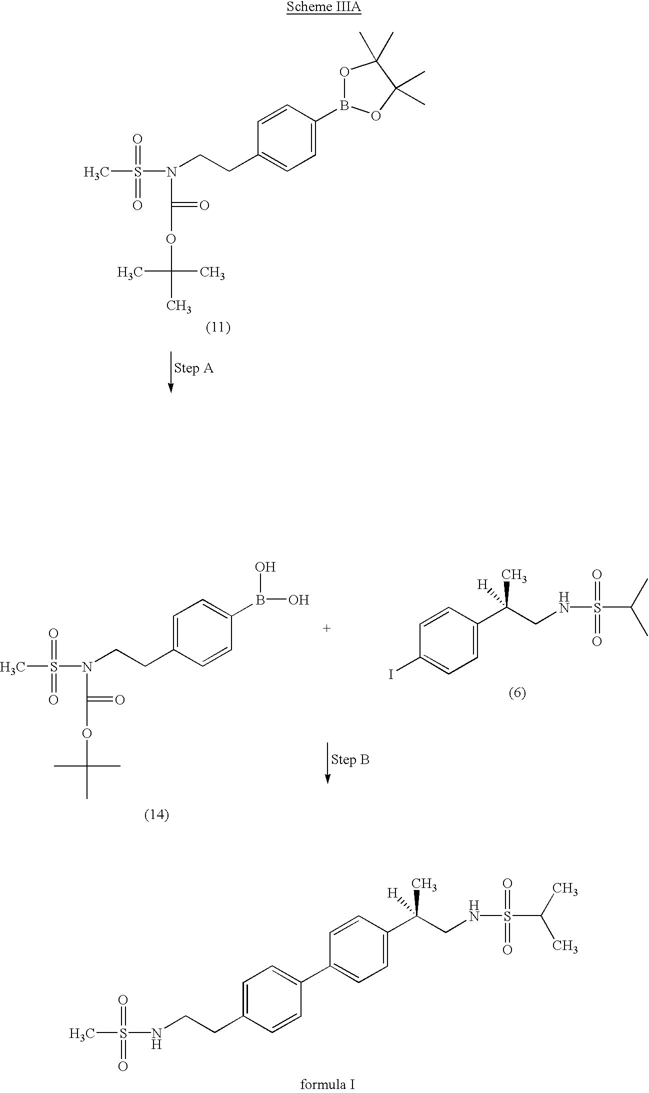

In Scheme IIIA, step A, compound (11) is subjected to boron pinacolate cleavage to provide the compound (14). For example, compound (11) is dissolved in a suitable organic solvent, such as acetone and added with stirring to an ammonium acetate solution to which an excess of sodium periodate has been added. The reaction mixture is allowed to stir for about 8 hours to about 16 hours and then it is concentrated under vacuum to remove the acetone. The aqueous phase is decanted from the oily product, and the aqueous is extracted with suitable organic solvents, such as methylene chloride and MTBE. The oily product and the organic extracts are combined and treated with aqueous base, such as sodium hydroxide, to provide a pH of about 12.5. The phases are separated and the organic phase is extracted with 1 N sodium hydroxide and water. The aqueous phase and aqueous extracts were then combined and washed with suitable organic solvents, such as methylene chloride and MTBE. The aqueous is then added to a suitable organic solvent, such as methylene chloride and treated with a suitable acid, such as 1 N sulfuric acid to provide a pH of about 3. The phases are separated and the aqueous phase is extracted with methylene chloride. The organic phase and organic extracts are combined and concentrated under vacuum. The residue is triturated with a suitable solvent mixture, such as MTBE/heptane to provide compound (14).

In Scheme IIIA, step B, compound (14) is coupled to compound (6) to provide the compound of formula I. For example compound (6) is combined with about 1.4 equivalents of compound (14) and about 1.2 equivalents of potassium carbonate in a suitable organic solvent, such as n-propanol. To this mixture is added water, and a catalytic amount of palladium (II) acetate. The reaction mixture is then heated at reflux for about 20 hours. It is then cooled to room temperature and diluted with a suitable organic solvent, such as ethyl acetate. The diluted mixture is filtered through Celite® which is rinsed with ethyl acetate. The filtrates are combined, concentrated under vacuum and the residue diluted with a suitable organic solvent, such as ethyl acetate and 10% aqueous potassium carbonate. The phases are separated and the aqueous phase is extracted with ethyl acetate. The organic phase and organic extracts are combined, dried over anhydrous magnesium sulfate, filtered, and partially concentrated. The solution is heated to about 60° C. with stirring and a suitable organic solvent, such as heptane is added to provide a ratio by volume for ethyl acetate/heptane of about 17:11. The solution is allowed to cool slowly to room temperature with stirring for about 8 hours to about 16 hours and then cooled to about 0° C. The resulting solids are collected by filtration and rinsed with ethyl acetate/heptane to provide the compound of formula 1.

The following examples are illustrative only and are not intended to limit the invention in any way. The reagents and starting materials are readily available to one of ordinary skill in the art. Unless indicated otherwise, the substituents are defined as hereinabove. As used herein, the following terms have the meanings indicated: “eq” refers to equivalents; “g” refers to grams; “mg” refers to milligrams; “ng” refers to nanograms; “L” refers to liters; “mL” refers to milliliters; “μL” refers to microliters; “mol” refers to moles; “mmol” refers to millimoles; “psi” refers to pounds per square inch; “min” refers to minutes; “h” refers to hours; “° C.” refers to degrees Celsius; “TLC” refers to thin layer chromatography; “HPLC” refers to high performance liquid chromatography; “GC” refers to gas chromatography; “Rf” refers to retention factor; “6” refers to part per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “aq” refers to aqueous; “iPrOAc” refers to isopropyl acetate; “EtOAc” refers to ethyl acetate; “EtOH” refers to ethyl alcohol; “MeOH” refers to methanol; “MTBE” refers to tert-butyl methyl ether; “DEAD” refers to diethyl azodicarboxylate; “DBU” refers to 1,8-diazabicyclo[5.4.0]undec-7-ene; “TMEDA” refers to N,N,N′,N′-tetramethylethylenediamine, and “RT” refers to room temperature.

### EXAMPLE 1

Preparation of {(2R)-2-[4-(4-{2-[(methylsulfonyl)amino]ethyl}phenyl)phenyl]propyl}[(methylethyl)sulfonyl]amine.

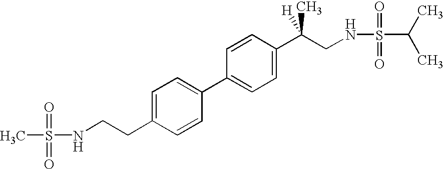

Preparation of 2-Phenyl-1-propylamine HCl.

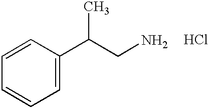

Scheme I, step A: To an autoclave hydrogenation apparatus under nitrogen was charged water-wet 5% palladium on carbon (453 g), ethanol (6.36 L), 2-phenylpropionitrile (636 g, 4.85 moles) and finally concentrated (12M) hydrochloric acid (613 g, 5.6 mole). The mixture was stirred rapidly and pressurized to 75-78 psi with hydrogen. The mixture was then heated to 50-64° C. for 3 hours. ¹H NMR analysis of an aliquot showed less than 5% starting material. The reaction mixture was depressurized and filtered to afford two lots of filtrate that were concentrated under reduced pressure to −400 mL each. To each lot was added methyl tert-butyl ether (MTBE) (2.2 L each) and the precipitate solids were allowed to stir overnight. Each lot was filtered and the collected solids were each washed with fresh MTBE (100 mL) and dried overnight. The lots were combined to afford 2-phenyl-1-propylamine HCl (634.4 g, 76.2%) as a white powder.

¹H NMR analysis of the free base: ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

Preparation of (2R)-2-phenylpropylamine Malate.

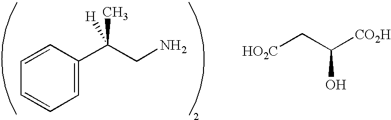

Scheme I, step B: To a dry 3-Liter round bottom flask under nitrogen was charged 2-phenyl-1-propylamine HCl (317.2 g, 1.85 moles), dry ethanol (2.0 L) and NaOH beads (75.4 g, 1.89 moles) that were washed in with additional ethanol (500 mL). The mixture was stirred for 1.6 hours, and the resulting milky white NaCl salts were filtered. An aliquot of the filtrate was analyzed by gas chromatography to provide the amount of free amine, 2-phenyl-1-propylamine, (1.85 moles). A solution of L-malic acid (62.0 g, 0.462 mole, 0.25 equivalents) in ethanol (320 mL) was added dropwise to the yellow filtrate and the solution was heated to 75° C. The solution was stirred at 75° C. for 30 minutes. The heat was removed and the solution was allowed to cool slowly. The resulting thick precipitate was allowed to stir overnight. The precipitate was filtered and dried under vacuum after rinsing with ethanol (325 mL) to afford (2R)-2-phenylpropylamine malate (147.6 g, 39.5%) as a white crystalline solid. Chiral GC analysis of the free base, 2-phenyl-1-propylamine revealed 83.2% e.e. enriched in the R-isomer (configuration was assigned via spectrometric comparison with commercial 2-phenyl-1-propylamine)

¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

A slurry of (2R)-2-phenylpropylamine malate (147.1 g, 83.2% e.e.) in 1325 mL ethanol and 150 mL deionized water was heated to reflux (-79.2° C.) until the solids went into solution. The homogeneous solution was allowed to slowly cool with stirring overnight. The precipitated white solids were cooled (0-5° C.) and filtered. The collected solids were rinsed with ethanol (150 mL) and dried at 35° C. to afford (2R)-2-phenylpropylamine malate (125.3 g, 85.2% recovery) as a white powder. Chiral GC analysis of the free base, (2R)-2-phenylpropylamine, revealed 96.7% e.e. enriched in the R-isomer.

¹H NMR (CD₃OD, 300 MHz) δ 7.32 (m, 10H), 4.26 (dd, 1H, J=3.6, 9.9), 3.08 (m, 6H), 2.72 (dd, 1H, J=9.3, 15.3), 2.38 (dd, 1H, J=9.3, 15.6), 1.33 (d, 6H, J=6.6).

Preparation of ((2R)-2-phenylpropyl)[(methylethyl)sulfonyl]Amine.

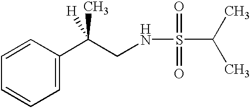

Scheme I, steps C and D: To a stirred slurry of (2R)-2-phenylpropylamine malate (200 g, 0.494 mol) in CH₂Cl₂ (1000 mL) was added 1.0 N NaOH (1050 mL, 1.05 moles). The mixture was stirred at room temperature for 1 hour and the organic phase was separated and gravity filtered into a 3.0 L round-bottom flask with a CH₂Cl₂ rinse (200 mL). The resulting free base, (2R)-2-phenylpropylamine, was dried via azeotropic distillation. Accordingly, the clear filtrate was concentrated to 600 mL at atmospheric pressure via distillation through a simple distillation head. Heptane (1000 mL) was added and the solution was concentrated again at atmospheric pressure to 600 mL using a nitrogen purge to increase the rate of distillation. The final pot temperature was 109° C.

The solution was cooled to room temperature under nitrogen with stirring to give a clear, colorless heptane solution (600 mL) of (2R)-2-phenylpropylamine. To this solution was added 4-dimethylaminopyridine (6.04 g, 0.0494 mol), triethylamine (200 g, 1.98 moles), and CH₂Cl₂ (500 mL). The mixture was stirred at room temperature until a clear solution was obtained. This solution was cooled to 5° C. and a solution of isopropylsulfonyl chloride (148 g, 1.04 moles) in CH₂Cl₂ (250 mL) was added dropwise with stirring over 2 hrs. The mixture was allowed to warm gradually to room temperature over 16 h. GC analysis indicated complete consumption of the (2R)-2-phenylpropylamine starting material.

The stirred mixture was cooled to 8° C. and 2 N HCl (500 mL) was added dropwise. The organic phase was separated and extracted with water (1×500 mL) and saturated NaHCO₃ (1×500 mL). The organic phase was isolated, dried (Na₂SO₄), and gravity filtered. The filtrate was concentrated under reduced pressure to provide ((2R)-2-phenylpropyl)[(methylethyl)sulfonyl]amine (230 g, 96%) as a pale yellow oil. ¹H NMR (CDCl₃, 300 MHz) δ 7.34 (m, 2H), 7.23 (m, 3H), 3.89 (br t, 1H, J=5.4), 3.36 (m, 1H), 3.22 (m, 1H), 3.05 (m, 1H), 2.98 (m, 1H), 1.30 (d, 3H, J=7.2), 1.29 (d, 3H, J=6.9), 1.25 (d, 3H, J=6.9).

Preparation of [(2R)-2-(4-iodophenyl)propyl][(methylethyl)sulfonyl]amine.

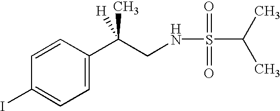

Scheme I, step E: A stirred room temperature solution of ((2R)-2-phenylpropyl)[(methylethyl)sulfonyl]amine (37.1 g, 0.154 mol) in glacial acetic acid (185 mL) was treated with concentrated H₂SO₄ (16.0 g, 0.163 mol), added dropwise in a slow stream, followed by a H₂O rinse (37 mL). To this solution (˜30° C.) was added H₅IO₆ (8.29 g, 0.0369 mol), followed by iodine (17.9 g, 0.0707 mol). The resulting reaction mixture was heated and allowed to stir for 3 h at 60° C. After HPLC analysis verified the consumption of starting material, the reaction mixture was cooled to 30° C. and a 10% aqueous solution of NaHSO₃ (220 mL) was added dropwise while maintaining the temperature between 25° C. and 30° C. The mixture crystallized to a solid mass upon cooling to 0-5° C.

The solids were suction filtered and rinsed with H₂O to afford 61.7 g of crude solids that were redissolved into warm MTBE (500 mL). This solution was extracted with H₂O (2×200 mL) and saturated NaHCO₃ (1×200 mL) and the organic phase was dried (MgSO₄), filtered, and concentrated under reduced pressure to −200 mL. Heptane (100 mL) was added dropwise to the product solution with slow stirring until crystallization commenced. An additional 100 mL of heptane was added and the resulting suspension was allowed to stir slowly overnight at room temperature. The mixture was then cooled (0° C.), filtered, and the collected solids were rinsed with heptane. The solids were then air-dried to afford the intermediate title compound, [(2R)-2-(4-iodophenyl)propyl][(methylethyl)sulfonyl]amine (33.7 g, 59.8%) as a white powder. Chiral Chromatography of this lot indicated 100% e.e.

¹H NMR (CDCl₃, 300 MHz) δ 7.66 (d, 2H, J=8.1), 6.98 (d, 2H, J=8.4), 3.86 (br t, 1H, J=5.1), 3.33 (m, 1H), 3.18 (m, 1H), 3.06 (m, 1H), 2.92 (m, 1H), 1.30 (d, 3H, J=6.6), 1.27 (d, 6H, J=6.6).

Preparation of (methylsulfonyl)(2-phenylethyl)amine.

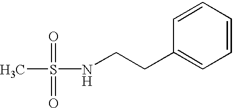

Scheme II, step A: To a 10° C. solution of phenethylamine (12.1 g, 0.100 mol) and triethylamine (11.1 g, 0.110 mol) in CH₂Cl₂ (50 mL) was added methanesulfonyl chloride (12.6 g, 0.110 mol) dropwise over 10 min. The solution was stirred at room temperature for 1.5 h and was then washed with 1 N HCl (5×20 mL). The organic phase was directly concentrated to provide the intermediate title compound, (methylsulfonyl)(2-phenylethyl)amine, (21.2 g, 93.3%) as an oil.

¹H NMR (CDCl₃, 300 MHz) 67.32 (m, 2H), 7.23 (m, 3H), 4.30 (br s, 1H), 3.40 (t, 2H, J=3.9), 2.88 (t, 2H, J=4.2), 2.81 (s, 3H).

Preparation of [2-(4-iodophenyl)ethyl](methylsulfonyl)amine.

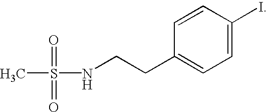

Scheme II, step B: To a stirring room temperature solution of (methylsulfonyl)(2-phenylethyl)amine (205 g, 1.03 moles), water (200 mL), 95% sulfuric acid (111 g, 1.08 moles) in acetic acid (1 L), was added iodine (111 g, 0.438 mol) and periodic acid (H₅IO₆, 45.6 g, 0.206 mol). The reaction mixture was warmed to 70-75° C. for 3 h. The heat was removed and the dark violet reaction mixture was allowed to proceed overnight at room temperature. Potassium hydroxide pellets (85%, 143 g, 2.16 moles) were added to neutralized the sulfuric acid and then enough saturated aqueous sodium sulfite was added to decolorize the mixture to afford a white suspension. The suspension was cooled to 15° C. and filtered. The filter cake was triturated thoroughly with water and was then dissolved in CH₂Cl₂ (1 L) and extracted with additional water (2×200 mL). The organic phase was concentrated under reduced pressure to provide the intermediate title compound, [2-(4-iodophenyl)ethyl](methylsulfonyl)amine, (201 g, 60.2%) as a white powder.

¹H NMR (CDCl₃, 300 MHz) δ 7.64 (d, 2H, J=4.8), 6.97 (d, 2H, J=5.1), 4.37 (br t, 1H, J=4), 3.36 (app. q, 2H, J=3.9), 2.85 (s, 3H), 2.82 (t, 2H, J=3.9).

Preparation of (tert-butoxy)-N-[2-(4-iodophenyl)ethyl]-N-(methylsulfonyl)carboxamide.

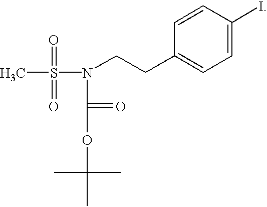

Scheme II, step C: A room temperature solution of [2-(4-iodophenyl)ethyl](methylsulfonyl)amine (201 g, 0.618 mol), 4-dimethylaminopyridine (3.8 g, 0.031 mol) and di-tert-butyl dicarbonate (162 g, 0.744 mol) in CH₂Cl₂ (1 L) was allowed to stir overnight. The reaction mixture was washed with water (2×400 mL) and the organic phase was concentrated to about 600 mL and hexanes (400 mL) was added. This combined solution was washed again with water (400 mL) and was concentrated to a solid that was suspended in hexanes (600 mL) and filtered. The collected solids were dried under reduced pressure to afford the intermediate title compound, (tert-butoxy)-N-[2-(4-iodophenyl)ethyl]-N-(methylsulfonyl)carboxamide (241.5 g, 91.5%) as a white solid.

¹H NMR (CDCl₃, 300 MHz) δ 7.63 (d, 2H, J=7.8), 6.98 (d, 2H, J=7.8), 3.88 (t, 2H, J=6.9), 3.10 (s, 3H), 2.88 (t, 2H, J=6.9), 1.51 (s, 9H).

Preparation of (tert-butoxy-N-(methylsulfonyl)-N-[2-[4-(4,4,5,5-tetramethyl(1,3,2-dioxaborolan-2-yl)phenyl]ethyl]carboxamide.

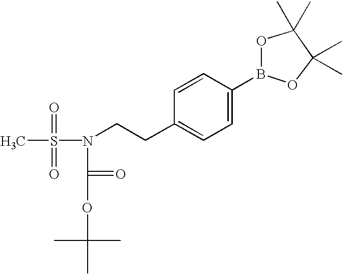

Scheme II, step D: To a degassed solution of (tert-butoxy)-N-[2-(4-iodophenyl)ethyl]-N-(methylsulfonyl)carboxamide (128 g, 0.300 mol), triethylamine (91.1 g, 0.900 mol), and 1,1′-bis(diphenylphosphino) ferrocenedichloropalladium (II)—CH₂Cl₂ complex (2.9 g, 0.0035 mol) in acetonitrile (600 mL) was added pinacolborane (50 g, 0.391 mol) dropwise. The mixture was stirred at 70-74° C. for 8 h and then was cooled to room temperature. The reaction mixture was concentrated to a fluid oil that was partitioned between MTBE (500 mL) and water (500 mL). The organic phase was separated and washed with water (2×200 mL) and concentrated to a residue that was partially dissolved with heptane (1 L). The heptane soluble fraction was filtered through Celite® 521 and concentrated to an oil (95 g). The residue was dissolved in acetone (600 mL) and heptane (600 mL) and filtered through Celite® 521. The combined filtrates were concentrated to 95 g of a mixture of a 3:1 molar ratio (¹H NMR, 81.0% by weight) of intermediate title compound, (tert-butoxy)-N-(methylsulfonyl)-N-{2-[4-(4,4,5,5-tetramethyl(1,3,2-dioxaborolan-2-yl))phenyl]ethyl}carboxamide, (60.3% potency corrected yield) and protio derivative.

¹H NMR (CDCl₃, 300 MHz) δ 7.75 (d, 2H, J=7.8), 7.23 (d, 2H, J=8.1), 3.87 (t, 2H, J-8.1), 2.99 (s, 3H), 2.90 (t, 2H, J=7.5), 1.53 (s, 9H), 1.33 (s, 6H), 1.27 (s, 6H).

Preparation of (Methylsulfonyl){2-[4-(4,4,5,5-tetramethyl(1,3,2-dioxaborolan-2-yl))phenyl]ethyl}amine.

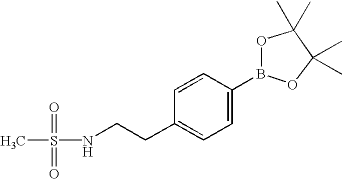

Scheme II, step E: To a 2 L flask charged with a stirring solution of (tert-butoxy)-N-(methylsulfonyl)-N-{2-[4-(4,4,5,5-tetramethyl(1,3,2-dioxaborolan-2-yl))phenyl]ethyl}carboxamide (98.7 g, 0.232 mol) in CH₂Cl₂ (500 mL) was added trifluoroacetic acid (82 mL, 121.4 g, 1.06 moles) dropwise from an addition funnel. No exotherm was observed and the reaction solution was allowed to stir at room temperature for 18 h.

HPLC analysis indicated 98% completion so the cooled (5° C.) reaction mixture was neutralized by the slow addition of 5N NaOH (175 mL). The pH of the aqueous phase was 10.5. The phases were separated and the aqueous phase was extracted with CH₂Cl₂ (50 mL). The combined CH₂Cl₂ phases were washed with brine (2×100 mL) and water (1×100 mL). The CH₂Cl₂ phase was diluted with heptane (300 mL) and was concentrated under reduced pressure to afford a suspension that was isolated by filtration. The collected solids were washed with pentane (2×100 mL) and dried under vacuum to provide the intermediate title compound, (methylsulfonyl){2-[4-(4,4,5,5-tetramethyl(1,3,2-dioxaborolan-2-yl))phenyl]ethyl}amine, (69.0 g, 91.4%) as a white powder.

¹H NMR (CDCl₃, 300 MHz) δ 7.77 (d, 2H, J=8.1), 7.22 (d, 2H, J=7.8), 4.26 (br t, 1H, J-6), 3.40 (q, 2H, J=6.9), 2.89 (t, 2H, J=6.6), 2.82 (s, 3H), 1.34 (s, 12H).

Preparation of 4-[2-[(methylsulfonyl)amino]ethyl]benzene Boronic Acid.

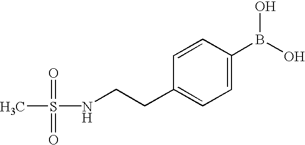

Scheme II, step F: (Methylsulfonyl){2-[4-(4,4,5,5-tetramethyl(1,3,2-dioxaborolan-2-yl))phenyl]ethyl}amine (68.0 g, 0.209 mol) was placed into a 2L flask and combined with acetone (600 mL), 1N ammonium acetate (600 mL), and NaIO₄ (168.1 g, 0.786 mol). This mixture was stirred at room temperature overnight. The reaction mixture was filtered to remove insoluble matter to afford filtrate A. The collected solids were washed with acetone (2×100 mL) and this filtrate was combined with filtrate A. The combined filtrates were concentrated under reduced pressure to 600 mL to afford a precipitate that was recovered by filtration. The collected solids were air-dried to give 110 g of crude material. This crude material was suspended in water (100 mL) and 5N NaOH was added until the pH was 12.5. The resulting suspension was filtered and the filtrate was treated with decolorizing carbon (Darco 6-60). The mixture was filtered and the filtrate was diluted with 10N H₂SO₄ until the pH was 5.0 to precipitate the intermediate title compound. This precipitate was collected by filtration and dried under reduced pressure to provide the intermediate title compound, 4-{(2-[(methylsulfonyl)amino]ethyl}benzene boronic acid, (41.9 g, 82.5%) as a white powder.

¹H NMR (acetone-d₆, 300 MHz) δ 7.82 (d, 2H, J=8.4), 7.27 (d, 2H, J=7.8), 7.11 (s, 2H), 6.03 (m, 1H), 3.36 (m, 2H), 2.91 (m, 2H), 2.84 (s, 3H).

Preparation of Final Title Compound.

Scheme III: An aqueous solution of potassium formate was prepared in the following manner. To 15 mL of water was added KOH (85% flakes, 6.73 g, 0.102 mol), then 98% formic acid (4.70 g, 0.102 mol). Alternatively, one may use commercially available potassium formate. To this solution was then added K₂CO₃ (2.76 g, 0.0210 mol), 4-{2-[(methylsulfonyl)amino]ethyl}benzene boronic acid (4.62 g, 0.190 mol), 1-propanol (100 mL), and [(2R)-2-(4-iodophenyl)propyl][(methylethyl)sulfonyl]amine (7.35 g, 0.200 mol). This mixture was deoxygenated via three vacuum/N₂-refill cycles. Palladium black (0.0215 g, 0.0002 mol) was added and the mixture was again deoxygenated via three vacuum/N₂-refill cycles. The reaction flask was heated in a preheated oil bath at 88° C. and the mixture was stirred overnight.

HPLC analysis showed complete consumption of 4-{2-[(methylsulfonyl)amino]ethyl}benzene boronic acid, and the mixture was diluted with ethyl acetate and filtered through Celite® to remove palladium. The mixture was concentrated under reduced pressure and the resulting residue was partitioned between ethyl acetate and water. The organic phase was concentrated and the solid residue was collected and recrystallized from 1:1 acetone/water to afford the final title compound, {(2R)-2-[4-(4-{2-[(methylsulfonyl)amino]ethyl}phenyl)phenyl]propyl}[(methylethyl)sulfonyl]amine, (6.2 g, 75%) as a white crystalline powder.

¹H NMR (CDCl₃, 300 MHz) δ 7.54 (dd, 4H, J=1.8, 8.1), 7.29 (dd, 4H, J=1.8, 8.1), 4.27 (t, 1H, J=6.6), 3.91 (m, 1H), 3.43 (q, 2H, J=6.6), 3.37 (dd, 1H, J=5.7, 7.5), 3.26 (m, 1H), 3.07 (m, 2H), 2.93 (t, 2H, J=6.6), 2.87 (s, 3H), 1.34 (d, 3H, J=7.2), 1.31 (d, 3H, J=6.9), 1.27 (d, 3H, J=6.6).

Additional Preparation of {(2R)-2-[4-(4-{2-[(methylsulfonyl)amino]ethy}phenyl)-phenyl]-propyl}[(methylethyl)sulfonyl]amine.

Scheme III: Within a single-neck, 3L round bottom flask equipped with a magnetic stir bar was placed potassium formate (112.8 g, 1.34 moles, 5.1 eq) and water (200 mL) to provide a pH 8 solution. Potassium carbonate (72.7 g, 0.526 mol, 2.0 eq), and 4-{2-[(methylsulfonyl)amino]ethyl}benzene boronic acid (60.8 g, 0.250 mol, 0.95 eq) was added to form a stirring suspension as 1-propanol (720 mL) was added. [(2R)-2-(4-iodophenyl)propyl][(methylethyl)sulfonyl]amine (96.6 g, 0.263 mol, 1.0 eq) was added followed by additional 1-propanol (600 mL). The resulting mixture was stirred for 3 minutes while the reaction flask was fitted with a heating mantle and a glycol-cooled reflux condenser. Vacuum (10-20 torr) was slowly applied to the system over 10 minutes. Stirring had stopped due to the additional precipitation of the cooled system; nevertheless, after 30 minutes, the system was returned to atmospheric pressure with nitrogen. With gentle heating, the flask was evacuated and refilled with nitrogen two additional times. Stirring was stopped and palladium black (0.28 g, 0.0026 mol, 0.01 eq) was quickly added to the flask.

Stirring was resumed and the system was again evacuated and returned to atmospheric pressure with nitrogen over a 2 minute cycle. This evacuation/nitrogen purge was repeated two more times over a 15 second cycle and the mixture was heated to reflux.

After 16 hours, an aliquot was removed and analyzed by HPLC (275 nm detection). Analysis showed 0.07% of achiral dimer, (methylsulfonyl){2-[4-(4-{2-[(methylsulfonyl)amino]ethyl}phenyl)phenyl]ethyl}amine, relative to the desired product, {(2R)-2-[4-(4-{2-[(methylsulfonyl)amino]ethyl}phenyl)phenyl]propyl}[(methylethyl)sulfonyl]amine. The reaction mixture was cooled to 50° C. and ethyl acetate (500 mL) was added. The reaction mixture was then cooled to room temperature and the product, {(2R)-2-[4-(4-{2-[(methylsulfonyl)amino]ethyl}phenyl)phenyl]propyl}[(methylethyl)sulfonyl]amine, began to precipitate. Additional ethyl acetate (IL) was introduced to redissolve the product and the upper organic phase was decanted and filtered through Celite® to remove palladium metal. The filter cake was rinsed with 1-propanol.

The homogeneous filtrate was concentrated under reduced pressure to remove n-propanol and after removal of 1.5 L of distillate, the product suspension was filtered. The combined filter cakes were dried to afford 109.8 g of crude final title compound.

Recrystallization: The crude final title compound (109.8 g) was dissolved in acetone (490 mL). This solution was filtered though a glass filter to retain a minor amount of dark insoluble material. To the slowly stirred filtrate was added water (300 mL) over 15 min. The resulting suspension was stirred for 16 minutes and additional water (20 mL) was introduced over 10 minutes. The suspension was subsequently stirred for 30 minutes at room temperature and was filtered. The cake was washed with 1:1 acetone/water (600 mL) and was dried at 35° C. overnight. This process afforded 80.3 g (81.1%) of {(2R)-2-[4-(4-{2-[(methylsulfonyl)amino]ethyl}phenyl)phenyl]propyl}[(methylethyl)sulfonyl]amine as a white crystalline powder with a mean particle size of about 29 to about 34 microns. HPLC analysis indicated 0.01% achiral dimer, (methylsulfonyl){2-[4-(4-{2-[(methylsulfonyl)amino]ethyl}phenyl)phenyl]ethyl}amine, and 0.02% chiral dimer, ((2R)-2-{4-[4-((1R)-1-methyl-2-{[(methylethyl)sulfonyl]amino}ethyl)phenyl]phenyl}propyl)[(methylethyl)sulfonyl]amine.

### EXAMPLE 2

Alternative Preparation of {(2R)-2-[4-(4-{2-[(methylsulfonyl)amino]ethyl}phenyl)phenyl]propyl}[(methylethyl)sulfonyl]amine.

Preparation of 4-{2-[-(tert-butoxy)N-(methylsulfonyl)carbonylamino]ethyl}benzene Boronic Acid.

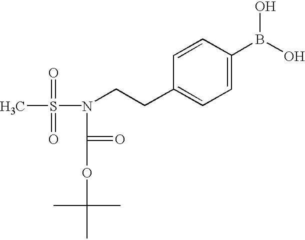

Scheme IIIA, step A: To a room temperature solution of (tert-butoxy)-N-(methylsulfonyl)-N-{2-[4-(4,4,5,5-tetramethyl(1,3,2-dioxaborolan-2-yl))phenyl]ethyl}carboxamide (81.0% potent, 95 g, 0.18 mol, prepared in example 1) in acetone (2 L) was added 1 N ammonium acetate (1L) and sodium periodate (145 g, 0.678 mol) with stirring. The reaction was allowed to proceed overnight. The reaction mixture was concentrated to remove the acetone, and the aqueous phase was decanted away from the oily product. The aqueous phase was extracted with CH₂Cl₂ (100 mL) and MTBE (2×100 mL). The combined oily product and organic phases were adjusted to pH 12.5 with the addition of 1 N NaOH. The phases were separated, and the organic phase was extracted with 1 N NaOH (100 mL) and water (2×100 mL). HPLC analysis (60% CH₃CN/40% H₂O, 2 mL/min, Zorbax C-18, 205 nm) of the organic phase indicated that the product had been removed from this phase. The aqueous phases (containing product) were finally combined and washed with CH₂Cl₂ (100 mL) and MTBE (2×100 mL). The aqueous phase was added to CH₂Cl₂ (450 mL) and 1 N H₂SO₄ was added until the aqueous phase was at pH 3.05. The phases were separated and the aqueous phase was extracted with CH₂Cl₂ (100 mL). The combined organic extracts (containing product) were concentrated to an oil (58.5 g) that crystallized overnight. The resulting solid mass was triturated with 10% MTBE in heptane (100 mL) to afford, after filtration and drying under reduced pressure, the intermediate title compound, 4-{2-[(tert-butoxy)-N-(methylsulfonyl)carbonylamino]ethyl}benzene boronic acid, (47.7 g, 77.2%) as a white powder.

¹H NMR (d₆-DMSO, 300 MHz) δ 7.83 (d, 2H, J=4.8), 7.24 (d, 2H, J=5.1), 7.12 (s, 2H), 3.90 (t, 2H, J=3.9), 3.12 (s, 3H), 2.95 (t, 2H, J=4.5), 1.52 (s, 9H).

Preparation of Final Title Compound.

Scheme IIIA, Step B: Run 1. Within a 3-neck, 1000 mL round-bottom flask was placed [(2R)-2-(4-iodophenyl)propyl][(methylethyl)sulfonyl]amine (15.0 g, 0.0408 mol, prepared in example 1), 4-{2-[(tert-butoxy)-N-(methylsulfonyl)carbonylamino]ethyl}benzene boronic acid (19.1 g, 0.0557 mol), K₂CO₃ (6.8 g, 0.0490 mol) and 1-propanol (300 mL). To this mixture was then added water (42 mL) and finally Pd(OAc)₂ (18 mg,-8.17×10⁻⁵ mol, 0.2 mol %). The resulting clear, pale amber solution was heated to reflux (87° C.) to become a dark amber, then a clear olive solution with stirring black particulates (Pd°). The reaction was allowed to stir for 20 h and was allowed to cool to room temperature. TLC analysis (1:9 EtOAc/CH₂Cl₂) of the resulting off-white suspension indicated desired product (R_{f} 032), complete consumption of [(2R)-2-(4-iodophenyl)propyl][(methylethyl)sulfonyl]amine (R_{f} 0.60) and only a trace of 4-{2-[(tert-butoxy)-N-(methylsulfonyl)carbonylamino]ethyl}benzene boronic acid (R_{f} 0.49). The suspension was diluted with EtOAc (300 mL) to give a clear, pale yellow solution that was filtered through Celite® (presaturated with EtOAc).

After washing the Celite® through with EtOAc, the filtrate was combined with that of an identical Run 2 which was conducted identically as described above. The combined filtrates from both runs were concentrated under reduced pressure to afford white solids that were diluted with EtOAc (1 L) and 10% K₂CO₃ (300 mL) to form a clear, amber biphasic solution that was agitated. The aqueous phase (light pink) was separated and the organic phase was washed with additional 10% K₂CO₃ (4×300 mL). The aqueous phase was back extracted with EtOAc (300 mL) and the combined organic phases (1500 mL) were dried (MgSO₄), filtered, and concentrated to a volume of about 620 mL within a 3 L round-bottom flask. The clear, pale yellow solution was stirred slowly while heating to 60° C. Heptane (400 mL) was added dropwise from a separatory funnel to the stirring EtOAc solution at 60° C. (17 volumes of EtOAc/11 volumes of heptane). The heptanes were added over a period of 1.5 h and the clear, pale yellow solution was allowed to cool slowly with slow stirring overnight. The resulting white crystalline solids were cooled to 0° C., filtered, and washed with a minimum of 1:1 EtOAc/heptanes to afford the final title compound, {(2R)-2-[4-(4-{2-[(methylsulfonyl)amino]ethyl}phenyl)phenyl]propyl)}[(methylethyl)sulfonyl]amine, (27.1 g, 75.7%) as a white crystalline powder.

### EXAMPLE 3

Alternative Preparation of ((2R)-2-phenylpropyl)[(methylethyl)sulfonyl]amine.

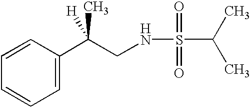

Preparation of (2R)-2-phenylpropan-1-ol.

An oven dried 500.0 mL three necked round bottom flask equipped with a mechanical stirrer, thermometer, addition funnel with a continuous nitrogen blanket is charged with 2.0 M solution of trimethylaluminum (65.6 mL, 131.2 mmol) and toluene (75.0 mL). Reaction solution was then chilled to −60° C. with dry ice/acetone bath. To this solution was then added R-styrene oxide dissolved in 100.0 mL of toluene over a period of 50.0 minutes (reaction is quite exothermic and can be controlled by the rate of addition of substrate). After stirring at this temperature for 60.0 minutes, reaction was brought to room temperature and stirred for 4.0 hours. Reverse quenched reaction at room temperature into a slurry of THF (100.0 mL) and sodium sulfate decahydrate (46.0 g) very cautiously over a period of 90.0 minutes (quenching was quite exothermic with evolution of gas). Filtered the precipitate formed over hyflo, then concentrated filtrate to provide the intermediate title compound, (2R)-2-phenylpropan-1-ol, (11.03 g, 92.6%) as an oil; ¹H nmr (CDCl₃) δ 1.28-1.29 (d, 3H, J=6.9 Hz), 1.5 (b, 1H), 2.9-3.0 (m, 1H), 3.69-3.70 (d, 2H, J=6.64 Hz), 7.24-7.35 (aromatic); ¹³C nmr (CDCl₃) δ 18.31, 43.15, 69.40, 127.38, 128.20, 129.26144.39.

Preparation of 2-((2R)-2-phenylpropyl)isoindoline-1,3-dione

An oven dried 250.0 mL three necked round bottom flask equipped with a mechanical stirrer, thermometer, addition funnel with a continuous nitrogen blanket is charged with (2R)-2-phenylpropan-1-ol (2.0 mL, 14.32 mmol), phthalimide (2.1 g, 14.32 mmol), triphenylphosphine (5.63 g, 21.48 mmol) and THF (70.0 mL). To this solution at room temperature was then added a solution of diethylazodicarboxylate (3.38 mL, 21.48 mmol) dissolved in THF (10.0 mL) over a period of 15-20 minutes (reaction exothermed slightly to 50° C. by the end of addition went from clear to reddish color). Stirred reaction to room temperature overnight). To the red solution was added water (50.0 mL) and the organic extracted with chloroform (140.0 mL). Dried the organic solution with anhydrous magnesium sulfate, filtered and concentrated under reduced pressure to an oil. To the oil was added heptane (150.0 mL) with stirring. Filtered of precipitates, then concentrated filtrate to an oil. Plug filtration of the oil over silica gel with 1:1 ethylacetate/hexane and concentrating product fractions afforded the intermediate title compound, 2-((2R)-2-phenylpropyl)isoindoline-1,3-dione, (4.27 g, 96%) as an oil which solidified on equilibrating to room temperature; ¹H nmr (CDCl₃) δ 1.3 (d, 3H), 3.34.0 (m, 1H), 3.7-3.9 (m, 2H), 7.1-7.3 (aromat. m, 2H), 7.63-7.7 (aromat. m, 2H), 7.8-7.85 (aromat. m, 4H).

Preparation of (2R)-2-phenylpropylamine.

A 500 mL three necked round bottom flask equipped with a mechanical stirrer, thermometer and addition funnel is charged with 2-((2R)-2-phenylpropyl)isoindoline-1,3-dione (11.54 g, 43.49 mmol), toluene (200.0 mL) and anhydrous hydrazine (2.73 mL, 86.99 mmol). Reaction is then stirred at room temperature for 3.0 hours and then heated at 90° C.-95° C. for 2.0 hours. Cooled the slurry to room temperature, filtered precipitates, then concentrated filtrate to provide the intermediate title compound, (2R)-2-phenylpropylamine, (5.58 g, 94.9%) an oil; ¹H nmr (CDCl₃) δ 1.21 (d, 3H), 1.40-1.60 (b, 2H), 2.68-2.80 (m, 1H), 2.81-2.87 (m, 2H) 7.20 (m, 2H), 7.32 (m, 2H).

Preparation of Final Title Compound.

To a solution of the (2R)-2-phenylpropylamine (1.2 g, 8.87 mmol) in hexane (16.0 mL) was added triethylamine (2.47 mL, 17.74 mmol) and dimethylaminopyridine (0.30 g, 2.47 mmol). Cooled reaction to 5° C., then added a solution of isopropylsulfonyl chloride (0.97 mL, 8.69 mmol) dissolved in methylene chloride (6.0 mL) over a period of 15.0 minutes. Stirred for 45.0 minutes, then stirred at room temperature for 120.0 minutes. Quenched reaction with 1 N HCl (20.0 mL) and extracted organic with methylene chloride (25.0 mL). Dried organic layer with anhydrous magnesium sulfate, filtered and concentrated filtrate to provide the final title compound, ((2R)-2-phenylpropyl)[(methylethyl)sulfonyl]amine, (1.93 g, 90.1%) an oil; ¹H nmr (CDCl₃) δ 1.25 (d, 3H, J=6.9 Hz), 1.29(d, 3H, J=6.9 Hz), 1.30 (d, 3H, J=7.2 Hz), 2.98 (m, 1H), 3.05 (m, 1H), 3.22 (m, 1H), 3.36 (m, 1H), 3.89 (b, 1H), 7.23 (m, 2H), 7.34 (m, 2H).

The ability of compounds of formula I to potentiate glutamate receptor-mediated response may be determined using fluorescent calcium indicator dyes (Molecular Probes, Eugene, Oreg., Fluo-3) and by measuring glutamate-evoked efflux of calcium into GluR4 transfected HEK293 cells, as described in more detail below.

In one test, 96 well plates containing confluent monolayers of HEK 293 cells stably expressing human GluR4B (obtained as described in European Patent Application Publication Number EP-A1-583917) are prepared. The tissue culture medium in the wells is then discarded, and the wells are each washed once with 200 μl of buffer (glucose, 10 mM, sodium chloride, 138 mM, magnesium chloride, 1 mM, potassium chloride, 5 mM, calcium chloride, 5 mM, N-[2-hydroxyethyl]-piperazine-N-[2-ethanesulfonic acid], 10 mM, to pH 7.1 to 7.3). The plates are then incubated for 60 minutes in the dark with 20 ΞM Fluo3-μM dye (obtained from Molecular Probes Inc., Eugene, Oreg.) in buffer in each well. After the incubation, each well is washed once with 100 μl, buffer, 200 μl, of buffer is added and the plates are incubated for 30 minutes.

Solutions for use in the test are also prepared as follows. 30 μM, 10 μM, 3 μM and 1 μM dilutions of test compound are prepared using buffer from a 10 mM solution of test compound in DMSO. 100 μM cyclothiazide solution is prepared by adding 3 μl, of 100 mM cyclothiazide to 3 ml of buffer. Control buffer solution is prepared by adding 1.5 μl DMSO to 498.5 μl of buffer.

Each test is then performed as follows. 200 pi of control buffer in each well is discarded and replaced with 45 μl of control buffer solution. A baseline fluorescent measurement is taken using a FLUOROSKAN II fluorimeter (Obtained from Labsystems, Needham Heights, Mass., USA, a Division of Life Sciences International Plc). The buffer is then removed and replaced with 45 μl of buffer and 45 μl of test compound in buffer in appropriate wells. A second fluorescent reading is taken after 5 minutes incubation. 15 μl of 400 μM glutamate solution is then added to each well (final glutamate concentration 100 μM), and a third reading is taken. The activities of test compounds and cyclothiazide solutions are determined by subtracting the second from the third reading (fluorescence due to addition of glutamate in the presence or absence of test compound or cyclothiazide) and are expressed relative to enhance fluorescence produced by 100 μM cyclothiazide.

In another test, HEK293 cells stably expressing human GluR4 (obtained as described in European Patent Application Publication No. EP-A1-0583917) are used in the electrophysiological characterization of AMPA receptor potentiators. The extracellular recording solution contains (in mM): 140 NaCl, 5 KCl, 10 HEPES, 1 MgCl₂, 2 CaCl₂, 10 glucose, pH=7.4 with NaOH, 295 mOsm kg-1. The intracellular recording solution contains (in mM): 140 CsCl, 1 MgCl₂, 10 HEPES, (N-[2-hydroxyethyl]piperazine-N 1-[2-ethanesulfonic acid]) 10 EGTA (ethylene-bis(oxyethylene-nitrilo)tetraacetic acid), pH 7.2 with CsOH, 295 mOsm kg-1. With these solutions, recording pipettes have a resistance of 2-3 MΩ. Using the whole-cell voltage clamp technique (Hamill et al.(1981)Pflügers Arch., 391: 85-100), cells are voltage-clamped at −60 mV and control current responses to 1 mM glutamate are evoked. Responses to 1 mM glutamate are then determined in the presence of test compound. Compounds are deemed active in this test if, at a test concentration of 10 pM or less, they produce a greater than 10% increase in the value of the current evoked by 1 mM glutamate.

In order to determine the potency of test compounds, the concentration of the test compound, both in the bathing solution and co-applied with glutamate, is increased in half log units until the maximum effect was seen. Data collected in this manner are fit to the Hill equation, yielding an EC₅₀ value, indicative of the potency of the test compound. Reversibility of test compound activity is determined by assessing control glutamate 1 mM responses. Once the control responses to the glutamate challenge are re-established, the potentiation of these responses by 100 μM cyclothiazide is determined by its inclusion in both the bathing solution and the glutamate-containing solution. In this manner, the efficacy of the test compound relative to that of cyclothiazide can be determined.

According to another aspect, the present invention provides a pharmaceutical composition, which comprises a compound of formula I or a pharmaceutically acceptable salt thereof and a pharmaceutically acceptable diluent or carrier.

The pharmaceutical compositions are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragcanth, gelatin, calcium silicate, micro-crystalline cellulose, polyvinylpyrrolidone, cellulose, water syrup, methyl cellulose, methyl and propyl hydroxybenzoates, talc, magnesium stearate, polyethylene glycol, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 5 micrograms to about 5 mg active ingredient, preferably about 5 micrograms to about 500 micrograms active ingredient, most preferably about 5 micrograms to about 200 micrograms active ingredient, and most especially preferably about 5 micrograms to about 100 micrograms. As used herein the term “active ingredient” refers to a compound included within the scope of formula I, such as {(2R)-2-[4-(4-{2-[(methylsulfonyl)amino]ethyl}phenyl)phenyl]propyl}[(methylethyl)sulfonyl]amine. The term “unit dosage form” refers to a physically discrete unit suitable as a unitary dosage for a patient, each unit containing a predetermined quantity of active ingredient calculated to produce the desired therapeutic effect, in association with a suitable pharmaceutical carrier, diluent, or excipient. The components of the formulation are brought together according to standard practice and procedures well known to one of ordinary skill in the art using conventional formulation and manufacturing techniques. The following formulation examples are illustrative only and are not intended to limit the scope of the invention in any way. The reagents and starting materials are readily available to one of ordinary skill in the art.

### Formulation

Hard Gelatin Capsules are Prepared Using the Following Ingredients to Provide Capsules Containing 0.005 mg, 0.040 mg, 0.200 mg, and 1.0 mg of {(2R)-2-[4-(4-{2-[(methylsulfonyl)amino]ethyl}phenyl)phenyl]propyl}[(methylethyl)sulfonyl]amine:

| | mg/ | mg/ | mg/ | mg/ |
|---|---|---|---|---|
| Component | capsule | capsule | capsule | capsule |
| {(2R)-2-[4-(4-{2- | 0.005 | 0.040 | 0.200 | 1.0 |
| [(methylsulfonyl)amino]ethyl} | | | | |
| phenyl)phenyl]propyl}[(methy | | | | |
| lethyl)sulfonyl]amine | | | | |
| PEG 3350 | 249.995 | 249.060 | 249.800 | 249.0 |
| Total | 250 | 250 | 250 | 250 |

As used herein the term “PEG” refers to polyethylene glycol. As used herein the term “suitable polyethylene glycol” refers to a polyethylene glycol that is a solid below a temperature of about 35° C. and allows dissolution of {(2R)-2-[4-(4-{2-[(methylsulfonyl)amino]ethyl}phenyl)phenyl]propyl}[(methylethyl)sulfonyl]amine when the suitable polyethylene glycol is in liquid form. Examples of suitable polyethylene glycols include PEG 3350, PEG 6000, PEG 8000, and the like. In addition, it is understood that a blend of PEG's falls within the scope of “suitable polyethylene glycol”, such as PEG 300 or PEG 400 being blended with a higher molecular weight PEG. Preferred suitable polyethylene glycols are PEG 3350, PEG 6000, PEG 8000, with PEG 3350 being most preferred. More specifically, for example, PEG 3350 is melted at a temperature of about 62° C. and {(2R)-2-[4-(4-{2-[(methylsulfonyl)amino]ethyl}phenyl)phenyl]propyl}[(methylethyl)sulfonyl]amine is added with stirring until there is complete dissolution. The molten solution is then filled directly into suitable capsules, such as hard gelatin capsules. The solution within the capsules hardens as it cools to room temperature.

The above formulation provides the necessary content uniformity at low doses of {(2R)-2-[4-(4-{2-[(methylsulfonyl)amino]ethyl}phenyl)phenyl]propyl}[(methylethyl)sulfonyl]amine.

In addition, by dissolving the compound in PEG, the generation of dust in the manufacturing process of the capsules is significantly reduced. As used herein the term “patient” refers to a mammal, such as a mouse, guinea pig, rat, dog, or human. It is understood that the preferred patient is a human.

The term “treating” (or “treat”) as used herein includes its generally accepted meaning which encompasses prohibiting, preventing, restraining, and slowing, stopping, or reversing progression of a resultant symptom. As such, the methods of this invention encompass both therapeutic and prophylactic administration.

As used herein the term “effective amount” refers to the amount or dose of the compound, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances. For example, a typical daily dose may contain from about 5 micrograms to about 5 mg of the active ingredient. The compounds can be administered by a variety of routes including oral, rectal, transdermal, subcutaneous, intravenous, intramuscular, bucal or intranasal routes. Alternatively, the compounds may be administered by continuous infusion.

We claim:

1. A compound of the formula: 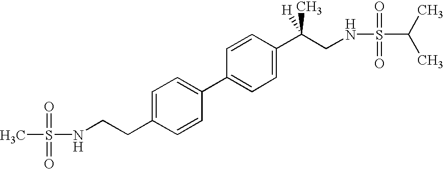
or a pharmaceutically acceptable salt thereof.

2. A compound of the formula:

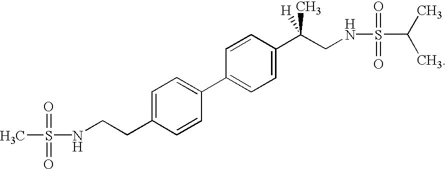

3. A composition comprising a compound of the formula: 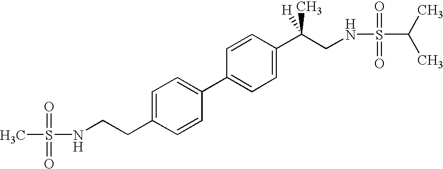
or a pharmaceutically acceptable salt thereof in combination with a pharmaceutically acceptable carrier, diluent or excipient.

4. A method of potentiating glutamate receptor function in a patient, which comprises administering to said patient an effective amount of a compound of the formula: 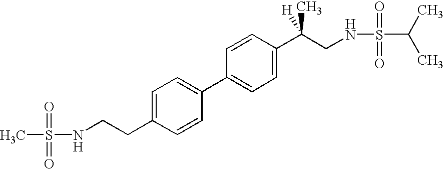
or a pharmaceutically acceptable salt thereof.

5. A method of treating depression in a patient comprising administering to said patient an effective amount of a compound of the formula: 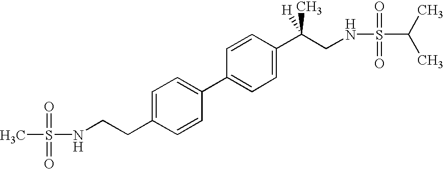
or a pharmaceutically acceptable salt thereof.

6. A method of treating schizophrenia in a patient comprising administering to said patient an effective amount of a compound of the formula: 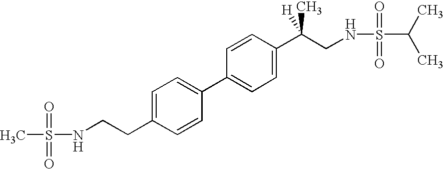
or a pharmaceutically acceptable salt thereof.

7. A method of treating cognitive disorders in a patient comprising administering to said patient an effective amount of a compound of the formula: 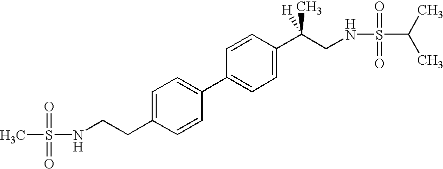
or a pharmaceutically acceptable salt thereof.

8. An article of manufacture comprising packaging material and a compound of formula: 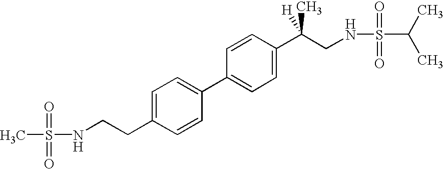
or a pharmaceutically acceptable salt thereof contained within said packaging material, wherein said packaging material comprises a label which indicates that said compound can be used for treating at least one of the following; Alzheimer's disease, schizophrenia, cognitive deficits associated with schizophrenia, depression, and cognitive disorders.

9. An article of manufacture according to claim 8 wherein said label indicates that said compound can be used for treating Alzheimer's disease.

10. An article of manufacture according to claim 8 wherein said label indicates that said compound can be used for treating schizophrenia.

11. An article of manufacture according to claim 8 wherein said label indicates that said compound can be used for treating depression.

12. An article of manufacture according to claim 8 wherein said label indicates that said compound can be used for treating cognitive deficits associated with schizophrenia.

13. A pharmaceutical composition comprising a compound of formula: 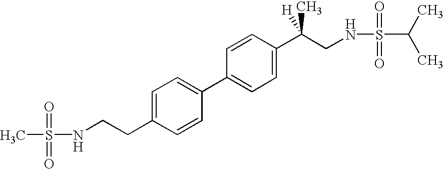
or a pharmaceutically acceptable salt thereof, in an amount of about 5 micrograms to about 500 micrograms.

14. A pharmaceutical composition according to claim 13 wherein the compound of formula: 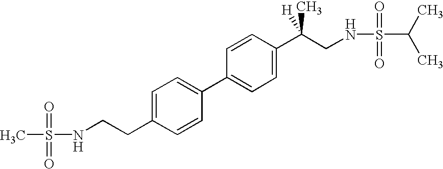
or a pharmaceutically acceptable salt thereof, is present in an amount of about 5 micrograms to about 200 micrograms.

15. A pharmaceutical composition according to claim 13 wherein the compound of formula: 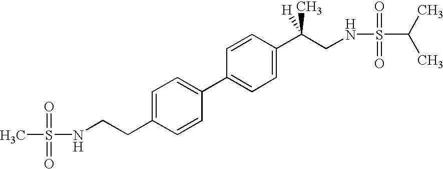
or a pharmaceutically acceptable salt thereof, is present in an amount of about 5 micrograms to about 100 micrograms.

16. A pharmaceutical composition prepared by a process comprising dissolving {(2R)-2-[4-(4-{2-[(methylsulfonyl)amino]ethyl}phenyl)phenyl]propyl}[(methylethyl)sulfonyl]amine in a suitable polyethylene glycol in liquid form, and then cooling the solution to room temperature.

17. A pharmaceutical composition according to claim 16 wherein the suitable polyethylene glycol is polyethylene glycol 3350.

18. A pharmaceutical composition according to claim 17 wherein the pharmaceutical composition is filled into suitable capsules.

19. A pharmaceutical composition according to claim 18 wherein the suitable capsules are hard gelatin capsules.

20. A pharmaceutical composition according to claim 18 wherein the {(2R)-2-[4-(4-{2-[(methylsulfonyl)amino]ethyl}phenyl)phenyl]propyl}[(methylethyl)sulfonyl]amine is present in an amount of about 5 micrograms to about 500 micrograms in each suitable capsule.

21. A pharmaceutical composition comprising, dissolving {(2R)-2-[4-(4-{2-[(methylsulfonyl)amino]ethyl}phenyl)phenyl]propyl}[(methylethyl)sulfonyl]amine and a suitable polyethylene glycol.

22. A pharmaceutical composition according to claim 21 wherein the suitable polyethylene glycol is PEG 3350.

23. A pharmaceutical composition according to claim 21 wherein the {(2R)-2-[4-(4-{2-[(methylsulfonyl)amino]ethyl}phenyl)phenyl]-propyl}[(methylethyl)sulfonyl]amine is present in an amount of about 5 micrograms to about 500 micrograms.

24. A pharmaceutical composition according to claim 21 wherein the {(2R)-2-[4-(4-{2-[(methylsulfonyl)amino]ethyl}phenyl)phenyl]propyl}[(methylethyl)-sulfonyl]amine is present in an amount of about 5 micrograms to about 500 micrograms and the suitable polyethylene glycol is PEG 3350.

25. Use of a compound of formula: 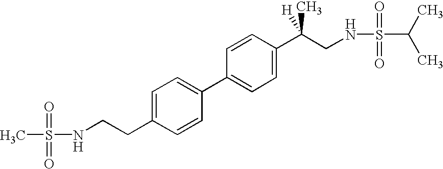
for the manufacture of a medicament for treating Alzheimer's disease.

26. Use of a compound of formula: 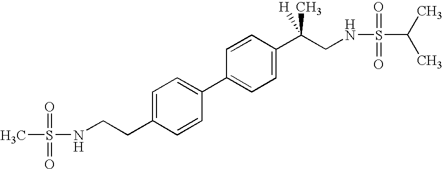
for the manufacture of a medicament for treating schizophrenia.

27. Use of a compound of formula: 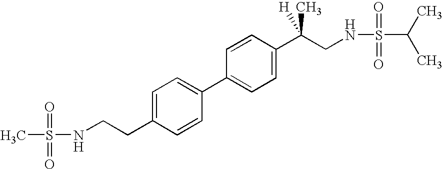
for the manufacture of a medicament for treating cognitive deficits associated with schizophrenia.

28. A compound of formula: 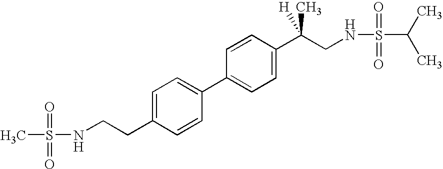
or a pharmaceutically acceptable salt thereof for use as a pharmaceutical.

29. A compound of formula: 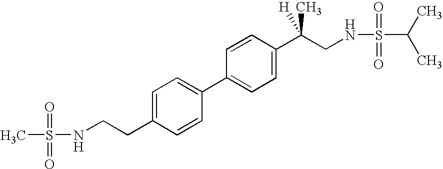
or a pharmaceutically acceptable salt thereof for the manufacture of a medicament for potentiating glutamate receptor function.
